(19) United States
(12) Patent Application Publication
(10) Pub. No.: US 2024/0268726 A1
(43) Pub. Date: Aug. 15, 2024

(54) REUSABLE GARMENTS AND PERSONAL CARE PRODUCTS WITH EMBEDDED DIAGNOSTICS

(71) Applicants: Anisha Roy, Cary, NC (US); Ishita Bafna, Morrisville, NC (US); Shailen Fofaria, Raleigh, NC (US); Nandini Kanthi, Cary, NC (US); Sophie Khullar, Apex, NC (US); Esther Ghim, Cary, NC (US)

(72) Inventors: Anisha Roy, Cary, NC (US); Ishita Bafna, Morrisville, NC (US); Shailen Fofaria, Raleigh, NC (US); Nandini Kanthi, Cary, NC (US); Sophie Khullar, Apex, NC (US); Esther Ghim, Cary, NC (US)

(21) Appl. No.: 18/441,614

(22) Filed: Feb. 14, 2024

Related U.S. Application Data
(60) Provisional application No. 63/485,006, filed on Feb. 15, 2023.

(51) Int. Cl. A61B 5/15; A61B 5/00; A61B 5/157; A61F 13/56; A61F 13/84; G01N 33/52

(52) U.S. Cl. CPC A61B 5/150045; A61B 5/150969; A61B 5/157; A61B 5/6804; A61F 13/5616; G01N 33/525; G01N 33/528; A61B 2562/0295; A61F 2013/8473; A61F 2013/8488

(57) ABSTRACT

Reusable garments and personal care products that have diagnostics embedded therein can be a 2-in-1 diagnostic and hygiene tool. In one example, a reusable menstrual pad with diagnostics embedded therein can serve as a low-cost detection tool for Human Papillomavirus (HPV) in developing regions. The pad, e.g., cloth-based sanitary napkin, can house a diagnostic mechanism so when menstrual blood hits the surface of the pad while in use, the mechanism—or testing strip—can process the blood to test it for HPV markers. The pad can address a complex problem by leveraging the monthly frequency of menstrual cycles to routinely test for HPV.

### CROSS-REFERENCES TO RELATED APPLICATIONS

This non-provisional application claims priority to and/or receives benefit from provisional application, titled “REUSABLE GARMENTS AND PERSONAL CARE PRODUCTS WITH EMBEDDED DIAGNOSTICS”, Ser. No. 63/485,006, filed on Feb. 15, 2023. The provisional application is hereby incorporated by reference in its entirety.

### TECHNICAL FIELD

The present disclosure generally relates to garments and personal care products, and, more specifically, to reusable garments and personal care products with embedded diagnostics.

### BACKGROUND

Whether it be from a surgery, a scraped knee, or a paper cut, we've all bled at least once in our lives. The menstrual cycle occurs 450 times in a woman's life; unfortunately, too much of the world has yet to normalize it. Menstrual blood is the only source of blood that is not traumatically induced, but primarily in developing nations, the stigma surrounding it has prevented proper access and maintenance of menstrual health for millions of menstruating people. Around 500 million individuals who menstruate are unable to access menstrual hygiene products, with over 65 percent of females in developing nations facing a similar fate.

Human Papillomavirus, or HPV, is the most common sexually transmitted infection in the world. HPV can cause abnormal tissue growth that can lead to cervical cancer (CC). In fact, the CDC stipulates that 91 percent of all cervical cancers are caused by a prior HPV infection. With respect to its impact, CC is the fourth most common malignancy seen in women globally (528,000 new cases every year) and the second most common in developing nations (445,000 new cases every year). Over 80% of the global CC burden occurs in underserved areas because early and accessible diagnostics are difficult to acquire, leading to over 230,000 annual deaths in these regions. Some countries—such as India and Ethiopia—offer either crude or no testing at all to screen for cervical cancer.

Evidently, a lack of resources seems to be the biggest roadblock to the timely detection of CC, yet even well-developed and well-resourced countries like the United States struggle when it comes to providing equitable access to diagnostics. Pap smears—where doctors collect cells from an individual's cervical wall and test them for CC markers—are supposed to be conducted every three years during a woman's annual checkup, yet around eight million menstruating people in the U.S. have not been screened in the past five years.

This is primarily because pap smears cost anywhere from $50 to $150 if they are paid for out-of-pocket, which is an expense that is seldom prioritized when the focus of our low-income population is to make rent and put food on the table. For them, every dollar counts, making affordability a huge point of contention for new innovations that aim to bridge this gap in healthcare.

$331 represents just for one pap smear and pelvic exam to screen for cervical disease. There are 385 additional proteins in menstrual blood than in systemic blood. Accuracy can be relatively high with proper diagnostic methods through menses.

### BRIEF DESCRIPTION OF THE DRAWINGS

The various advantages and features of the present technology will become apparent by reference to specific implementations illustrated in the appended drawings. A person of ordinary skill in the art will understand that these drawings show only some examples of the present technology and would not limit the scope of the present technology to these examples. Furthermore, the skilled artisan will appreciate the principles of the present technology as described and explained with additional specificity and detail through the use of the accompanying drawings in which:

FIG. 1 is a top view of an exemplary personal care product having embedded diagnostics therein, according to some aspects of the disclosed technology;

FIG. 2 is a front view of an exemplary personal care product also illustrated in FIG. 1, according to some aspects of the disclosed technology;

FIG. 3 is a top view of an exemplary personal care product having embedded diagnostics therein, according to some aspects of the disclosed technology;

FIG. 4 is an exploded perspective view of an exemplary personal care product also illustrated in FIG. 3, according to some aspects of the disclosed technology;

FIG. 5 is a diagram illustrating an exemplary lateral flow immunoassay strip, according to some aspects of the disclosed technology;

FIG. 6 is a diagram illustrating an exemplary cover over the lateral flow immunoassay strip, according to some aspects of the disclosed technology;

FIGS. 7A-C are top views of exemplary personal care products having different sizing, according to some aspects of the disclosed technology; and

FIGS. 8A-B illustrates integration of diagnostics into a garment, according to some aspects of the disclosed technology.

FIGS. 9-12 illustrate different views of a garment having diagnostics embedded therein, according to some aspects of the disclosed technology.

FIG. 13 illustrates an exploded side view of layers used in a garment having embedded diagnostics therein, according to some aspects of the disclosed technology.

FIG. 14 illustrates a top view of embedded diagnostics and display of diagnostics results, according to some aspects of the disclosed technology.

FIG. 15 illustrates a top view 1500 of embedded diagnostics and display of diagnostics results and a part of the layered assembly 902, according to some aspects of the disclosed technology.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a more thorough understanding of the subject technology. However, it will be clear and apparent that the subject technology is not limited to the specific details set forth herein and may be practiced without these details. In some instances, structures and components are shown in block diagram form to avoid obscuring the concepts of the subject technology.

### Overview

Human Papillomavirus (HPV) causes abnormal tissue growth that can lead to cervical cancer. In fact, 91% of all cervical cancers were caused by a prior HPV infection. And, while cervical cancer is very preventable, approximately 80% of deaths from it occur in underserved regions. This is because these areas lack the healthcare capital to fund sanitary lab facilities and highly trained personnel to conduct extensive diagnostics. So, developing countries need a more cost-effective and user-friendly way to screen for HPV. Many solutions fail to address inaccessibility and unaffordability.

One solution involves a self-administered HPV test where users are provided a kit to swab their vaginal tissue for a sample. This sample is then shipped to a lab that processes the specimen and, ultimately, determines whether or not the user has HPV. For some populations, at-home tests have been a more available and effective solution if access to clinics is difficult; however, they present two key issues in terms of usability. Firstly, users must perform a slightly invasive procedure to actually collect the vaginal sample, which can be both uncomfortable and painful if done incorrectly. Secondly, self-administering this test requires a certain amount of education to increase knowledge and overall willingness to use the product, which is especially difficult to accomplish amongst populations in developing countries because of language barriers and social stigma associated with invasive screenings. Many products and services require a lot of understanding to implement and access (e.g., at-home tests require that users consult a diagram when swabbing themselves, some at-home tests require users to separate the testing strip from the pad and package it without contaminating the sample, etc.) At-risk individuals who struggle to afford and acquire diagnostic resources can benefit from a streamlined product that they can operate without getting confused.

One solution involves non-reusable pads that contain removable testing strips that are separately sent to labs for testing. In other words, these one-time use non-reusable pads generate additional garbage. In regions that lack proper waste disposal infrastructure (e.g. Cambodia, Philippines, etc.), this can be extremely problematic. Even if one more piece of plastic ends up in a landfill, that is one more piece of waste that contributes to the millions of deaths caused by plastic pollution. Much of the waste is either discarded in our oceans, burned in landfills, or dumped in villages located within developing countries, killing upwards of one million individuals every year.

The efficacy of these solutions is also contingent on users' access to labs that would process their samples. In poorly resourced communities that lack shipping infrastructure and nearby laboratory facilities, users would struggle to access their results in a timely manner. Moreover, laboratory testing requires physical labor that constitutes higher costs for these diagnostic products. This means that the low-income and isolated populations that would actually benefit from easy-to-use diagnostics cannot access these products.

Another solution involves using an electrochemical detection system (EDS) to screen for CC markers. This solution has several shortcomings: access to power, technology, and internet connections for users to interpret data, contains non-reusable materials which increases waste, employs expensive electrochemical devices, and use of plastic-based substrates that are not eco-friendly.

Broadly, many solutions have these problems. Solutions are expensive. Regular screenings are expensive to get frequently. Medical and personal technologies are costly and inaccessible. Manufacturing costs for many medical products are high. Solutions require complicated infrastructure. Conducting diagnostics requires highly trained personnel, well developed labs to process results, and sanitary facilities. This is often not accessible in developing regions. Solutions are invasive. Current methods of cervical screening, including pap smears and blood drawings, require needles or painful methods to extract samples from the body. The risk of infection increases in regions with low sanitation.

A reusable menstrual pad with diagnostics embedded therein can serve as a low-cost detection tool for HPV in developing regions. The pad, e.g., cloth-based sanitary napkin, can house a diagnostic mechanism so when menstrual blood hits the surface of the pad while in use, the mechanism—or testing strip—can process the blood to test it for HPV markers. If the person has HPV, their pad will change colors to display two horizontal red lines. Menstrual blood was the ideal choice to test as it can be 94% accurate at diagnosing HPV. Additionally, the pad can be lined with a charcoal fiber fabric to prevent 70% bacterial growth and facilitate the longevity and hygiene of the pad. The pad can address a complex problem by leveraging the monthly frequency of menstrual cycles to routinely test for HPV.

In some embodiments, the diagnostics are embedded in a reusable and washable menstrual pad form. The menstrual pad may include several layers, including a hydrophobic translucent organdy fabric, superabsorbent microfiber fabric, charcoal bamboo fiber, and waterproof Polyurethane Laminate (PUL) layer. The layers may form a layered assembly. The menstrual pad may include wings and wing clasps. The menstrual pad can be washed and reused. In some embodiments, the diagnostics are removable from the menstrual pad. In some embodiments, the diagnostics are not intended to be removed from the menstrual pad. The diagnostics may include a lateral flow assay fabric strip. The lateral flow assay fabric strip may be partially covered by the translucent organdy fabric while leaving a blood sample pad exposed for collecting menses. The diagnostics, e.g., a test strip, may include a collection pad, nanotubing filled with a buffer, a sample pad, a conjugate pad, and a wick pad.

In some embodiments, the diagnostics are embedded in a reusable and washable menstrual pad form. The menstrual pad may include several layers, including a transparent polymer layer, a surface layer, absorbent layer, waterproof layer, and anti-microbial backing. The layers may form a layered assembly. The menstrual pad may include wings and wing clasps. The menstrual pad can be washed and reused. The diagnostics may include a test strip, e.g., a lateral flow assay test strip. The test strip may be partially covered by transparent polymer to embed the test strip on, in, or under the surface layer while leaving a collection pad exposed for collecting menses. The test strip may include a collection pad, nanotubing filled with a buffer, a sample pad, a conjugate pad, and a wick pad. Results can be read through the transparent polymer layer. The menstrual pad may come in different sizes. In some embodiments, the diagnostics are removable from the menstrual pad. In some embodiments, the diagnostics are not intended to be removed from the menstrual pad. In some embodiments, the structures implemented in the menstrual pad form may be provided in a wearable garment such as underwear.

In some embodiments, the diagnostics are embedded in a reusable and washable garment that can be worn by a user. The garment may include a layered assembly that covers a user from the front of the user just below the abdomen to the back of the user up to the abdomen. The garment may include wings and wing clasps. The garment includes hip straps that wraps a user on the left side and the right side of the hip. The hip straps are affixed to the layered assembly. Sizing of the garment may be adjusted by adjusting the length of hip straps by adjusting a slide adjustor. Diagnostics are embedded in the layered assembly. The layered assembly may include a transparent polymer layer, a surface layer, a distribution layer, an absorbent layer, a waterproof layer, and anti-microbial coating/backing. The garment can be washed and reused. The diagnostics may include a test strip, e.g., a lateral flow assay test strip. The test strip may be partially covered by transparent polymer to embed the test strip on, in, or under the surface layer while leaving a collection pad exposed for collecting menses. The test strip may include a collection pad, nanotubing filled with a buffer, a sample pad, a conjugate pad, and a wick pad. In some embodiments, the test strip may include a lateral flow assay mechanism. In some embodiments, the diagnostics are removable from the menstrual pad. In some embodiments, the diagnostics are not intended to be removed from the menstrual pad.

### Exemplary Reusable Garments and Personal Care Products That Have Diagnostics Embedded Therein

Reusable garments and personal care products that have diagnostics embedded therein can be a 2-in-1 diagnostic and hygiene tool. The solution can maximize accessibility of HPV tests by utilizing a commonly stigmatized indicator: menstrual blood. Accessibility means eradicating financial barriers and eliminating the diagnostic industry's standard reliance on laboratory infrastructure. In medical research, menstrual blood can be 87.5% accurate at detecting HPV on the first day of the cycle. The most common strain of HPV is HPV-16 and it serves as a precursor for 65% of cervical cancer cases. Specifically, if the user has HPV, they are at risk of developing abnormal cytology. This causes their body's viral proteins to mutate into E6 oncoproteins (or in some cases, HPV 16/18 proteins), which are the biological markers that our device can be designed to detect.

Throughout the pandemic, COVID-19 rapid tests were an easy way for people to test whether or not they had the virus from the comfort of their own home. Similar to pregnancy tests, some diagnostic products also allowed users to view their test results in a matter of minutes without having to mail their sample to a lab. These products use a lateral flow immunoassay (LFA).

To enable users to be able to test for HPV at home while addressing some of the concerns described herein, a reusable menstrual pad is constructed to embed an LFA strip therein. The menstrual pad is reusable and washable. The LFA strip can effectively collect a sample of menses while it is embedded in the pad, e.g., on the first day of the user's menstrual cycle. The user can readily interpret the results on the LFA strip.

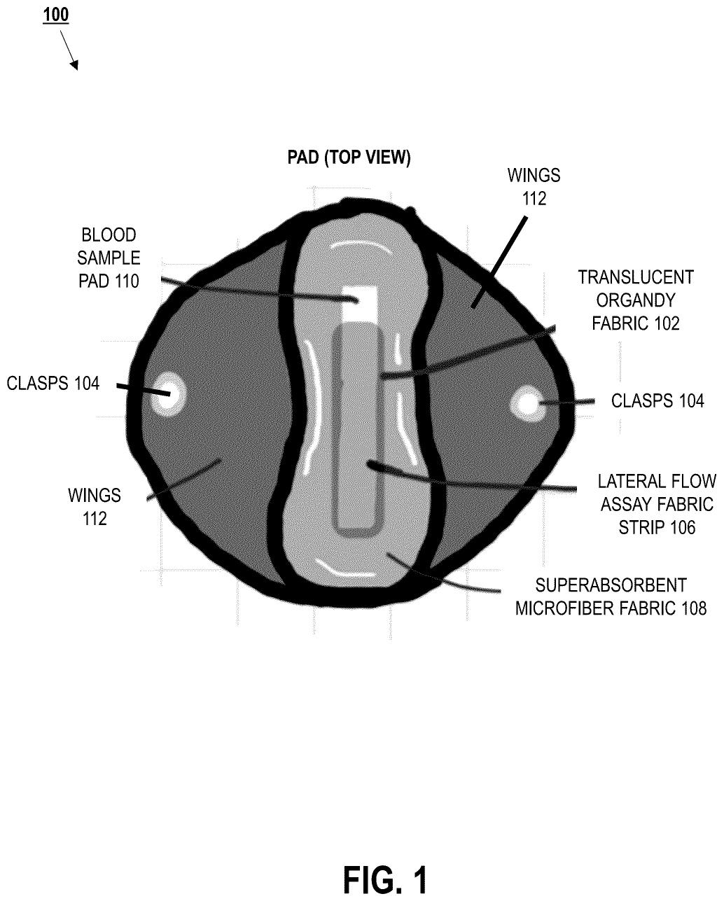

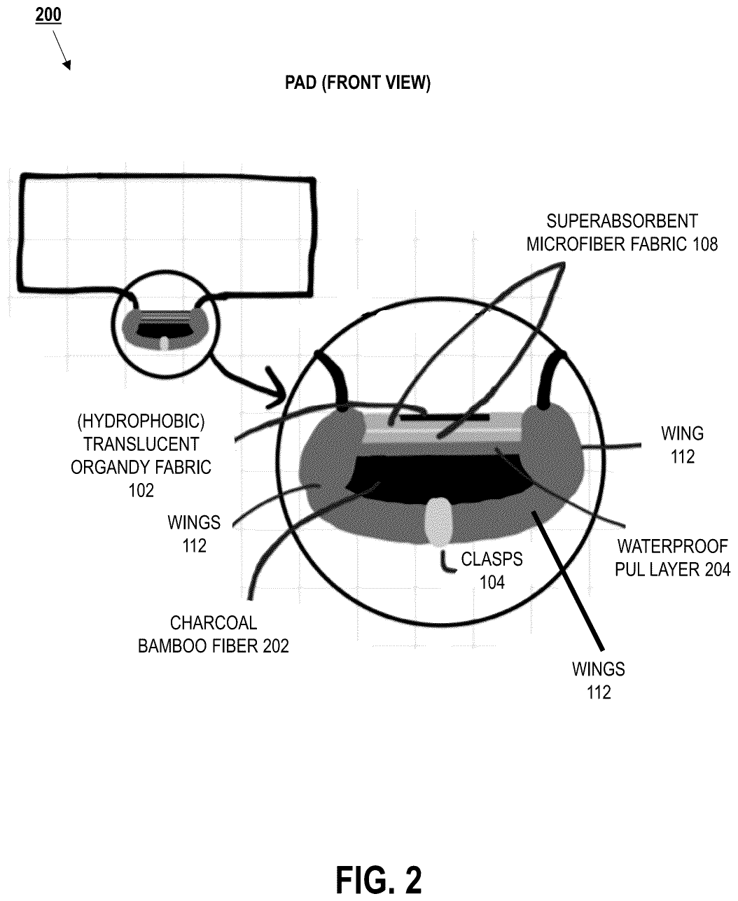

FIG. 1 is a top view 100 of an exemplary personal care product, in this case, a menstrual pad, having embedded diagnostics therein, according to some aspects of the disclosed technology. FIG. 2 is a front view 200 of the exemplary personal care product also illustrated in FIG. 1, according to some aspects of the disclosed technology. The personal care product may include (hydrophobic) translucent organdy fabric 102, clasps 104 (e.g., one or more buttons), lateral flow assay fabric strip 106, superabsorbent microfiber fabric 108, blood sample pad 110, wings 112, charcoal bamboo fiber 202, and waterproof PUL layer 204. The LFA strip illustrated in FIGS. 1 and 2 can include four main sections: the sample pad, the conjugate pad, the nitrocellulose membrane, and the absorbent pad. In some cases, the pad design involves covering the entire testing strip except the sample pad so that any additional menstrual blood that lands on the strip will not interfere with the mechanism itself. The sample pad may be exposed (e.g., not covered by translucent organdy fabric 102) for the purpose of absorbing the user's menstrual blood onto the strip and transporting it down the strip via capillary action. The blood then moves onto the conjugate pad, which can be coated with conjugated anti-E6 antibodies and colloidal gold nanoparticles. Antigens in the blood sample can then bond with the conjugated antibodies before flowing to the nitrocellulose membrane. The membrane may allow for complexes to be visualized, therefore identifying analytes, in this example, HPV 16/18 proteins or E6 oncoproteins, within a liquid blood sample. If the E6 is present in the blood, the bound antigen and conjugated antibody pair will bind with target antigens located on the test line (see diagram). The colloidal gold/color reagent can be activated when the complexes are bound to the test line, causing a colored line to appear on the strip. Thereafter, leftover viral-protein antibody complexes flow to the control line and present a similar color change. The control line may act as a “second trial” to confirm that the results are accurate. A test can be considered positive when both lines indicate a color change. Following the nitrocellulose membrane, the remainder of the sample may flow to the absorbent pad. This portion of the strip can prevent the sample from clogging in the strip and moving leftover, or unbound, proteins out of the way to avoid unintentional sensitivity. Moreover, the LFA technique is favored for its affordability, adaptability, and speed, making it an excellent candidate for cheap at-home HPV tests. The results may be read through translucent organdy fabric 102. In some cases, a layer of material may be parchmented or parchmentized to make the material transparent to form a translucent layer. Such a translucent layer may be used as translucent organdy fabric 102. This also allows the user to easily read the testing strip. Parchmentizing can include a finishing treatment to impart stiffness and a degree of translucency to a material such as fabric.

The pad shown in FIGS. 1-2 not only houses a diagnostic mechanism, but it also accommodates the mechanism into a sanitary product. This combination enhances user compatibility with the device, facilitating at-home testing regardless of access to education, sanitation, or reproductive health support. The menstrual pad can be made from cloth, making it washable and reusable. Absorbent microfiber fabrics can line the underside of the pad for its durability, soft texture, comfort, and absorbance. Beneath the fabrics, a layer of polyurethane laminate (PL) can waterproof and sterilize the pad. In other words, the PL layer can help to prevent leakage and facilitates longevity. The underside and wings of the pad can be covered with a charcoal fiber fabric that is 70% effective in inhibiting bacterial growth. The charcoal fiber fabric can combat poor sanitation and ensure protection of the testing strip, knowing that it could be prone to degradation or enhanced sensitivity if exposed to other natural elements. Additionally, as an at-home testing mechanism, it is important that the menstrual pad is comfortable and hygienic for the user to wear like they would any other menstrual product.

The construction and materials of the pad in FIGS. 1-2 takes into consideration a variety of factors with respect to feasibility, usage, accuracy, and cultural impact. In some embodiments, the pad can be used over many menstrual cycles. In some embodiments, the pad can be used as a reusable pad that is used over a single menstrual cycle (e.g., 3-7+ days). Menstrual blood may have the highest accuracy in detecting HPV on the first day of the menstrual cycle, so ideally, users would use the pad at the beginning of their menstrual cycle. The diagnostic mechanism itself is not reusable, and each time a user wants to test themselves, a new diagnostic mechanism may be inserted into the reusable pad. The pad itself, however, is reusable, so users can simply wash the product and re-wear it for the remainder of their cycle and future cycles. The layers of protective fabrics ensure that the pad is protected and safe for users when worn or used

The pad having the diagnostic method embedded therein in FIGS. 1-2 can leverage the monthly frequency of menstrual cycles to routinely screen for HPV. This is especially crucial for populations that are at risk for the spread of STDs through frequent nonconsensual sexual relations. The pad advantageously conceals the testing mechanism to curb any potential distrust users may have in the product or stigma against feminine sexual health, especially in countries where populations are not extensively educated about sex.

Positive tests can lead to anxiety for users and families. It may be important that the test is concerned with only HPV cases that are higher risk for cervical cancer to ensure that positive tests do not cause anxiety or propagate unnecessary expenses for extensive diagnostics in under-resourced regions. In some cases, the pad in FIGS. 1-2 examines HPV-16 E6 oncoproteins, only, as they are the markers that are most commonly associated with fatal cervical cancers. Nevertheless, a positive HPV test can be crucial in warning patients that they may possess a potentially life-threatening STD. If a test is considered positive, users can then take the next step of finding a physician for proper diagnosis and care before their condition exacerbates into something deadly and expensive to treat.

The pad in FIGS. 1-2 do not rely on inefficient diagnostic pipelines (e.g., sanitary lab facilities, highly trained personnel) that are usually needed to conduct extensive diagnostics. Also, the pad in FIGS. 1-2 do not rely on the use of complicated and expensive electrochemical sensors and readers. Moreover, the pad in FIGS. 1-2 can be extended and modified to perform diagnostics for other disorders.

The pad in FIGS. 1-2 operates at the pinnacle of 3 “pros”. The pad can pro-vide an effective solution for the under-resourced, pro-tect menstruating people from HPV and cervical cancer, and allow users to pro-sper with increased access to menstrual resources. The HPV diagnostic tool acts as a gateway for providing people with a long term, reusable menstrual pad. The pad can address cervical cancer fatalities and period poverty in developing nations all while mitigating the obstacles of cost, infrastructure, and sanitation to empower menstruating people with the assurance that their sexual health is valued.

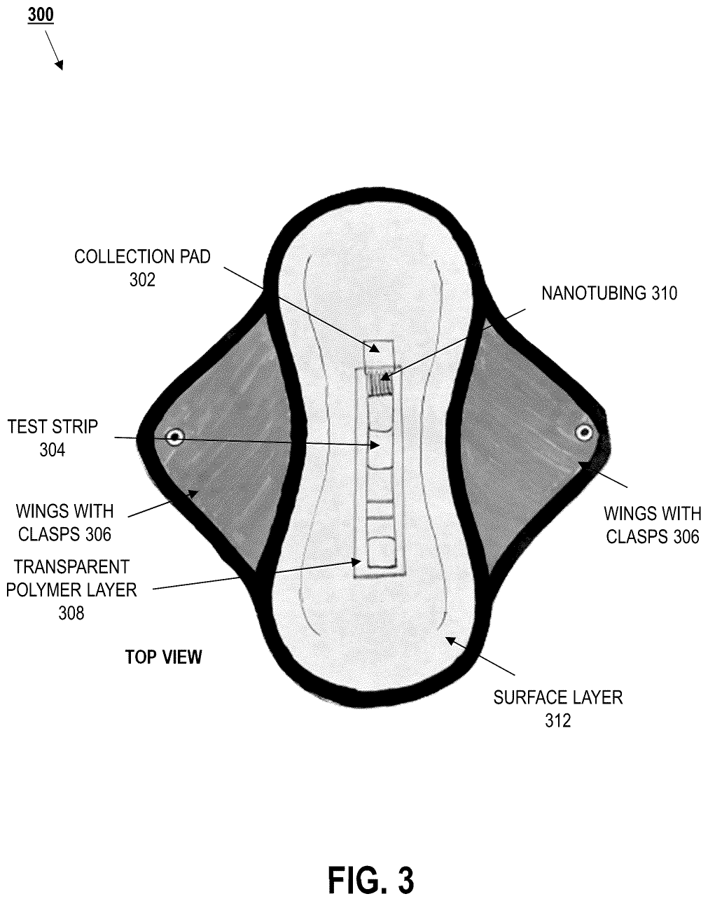

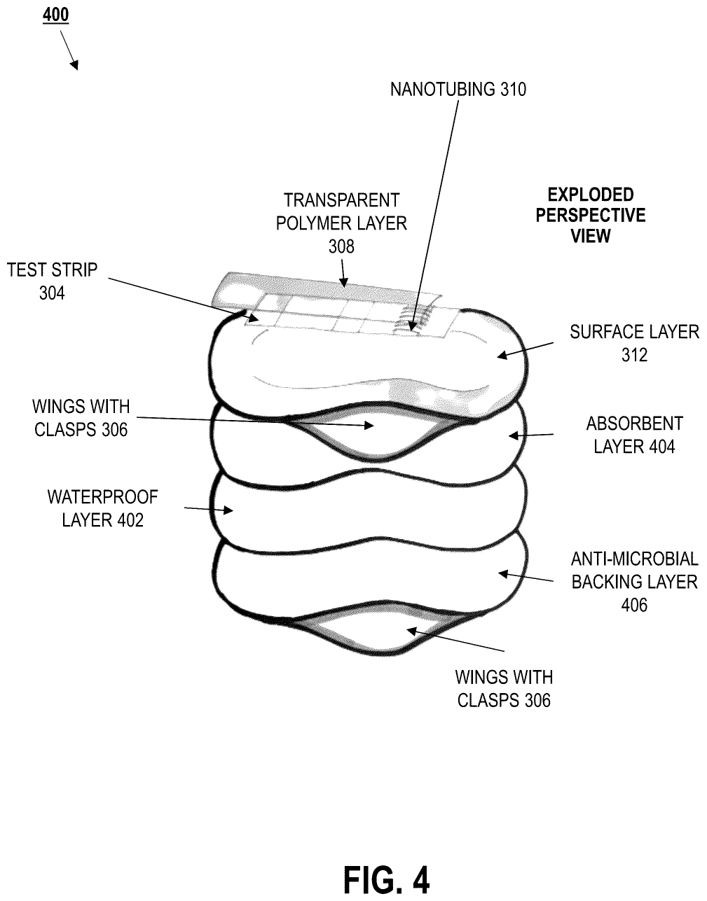

FIG. 3 is a top view 300 of an exemplary personal care product, in this case, a menstrual pad, having embedded diagnostics therein, according to some aspects of the disclosed technology. FIG. 4 is an exploded perspective view 400 of the exemplary personal care product also illustrated in FIG. 3, according to some aspects of the disclosed technology. The pad seen in FIGS. 3-4 is similar to the pad illustrated in FIGS. 1-3, but with some optional modifications and/or additional aspects.

The pad may include transparent polymer layer 308, surface layer 312, absorbent layer 404, waterproof layer 402, and anti-microbial backing layer 406. The pad may include wings with clasps 306. Wings with clasps may be formed from surface layer 312 and anti-microbial backing layer. The pad has a test strip 304 embedded therein.

The test strip 304 has at least a collection pad 302 and nanotubing 310. The collection pad 302 and nanotubing 310 (e.g., nanotubes) can allow for the entirety of the analyses to occur on the menstrual pad. The strip 304, except for the collection pad 302, may be covered by the transparent polymer layer 308.

The transparent polymer layer 308 may be made from polyisoprene, which is a hydrophobic polymer, to prevent any interference with the testing mechanism. This also allows the user to easily read the testing strip.

One method of using the pads and/or garments described herein includes:
A user wears pad on day 1 of menstrual cycle,
Washing or wipe surface of pad when full,
Looking through transparent polymer layer for test results, such as two blue lines,
Thoroughly wash pad,
Rewear pad on a subsequent day of menstrual cycle, and
After washing pad, the test results would not be valid, but pad can be reused, e.g., for additional days of the menstrual cycle and further menstrual cycles not requiring testing.

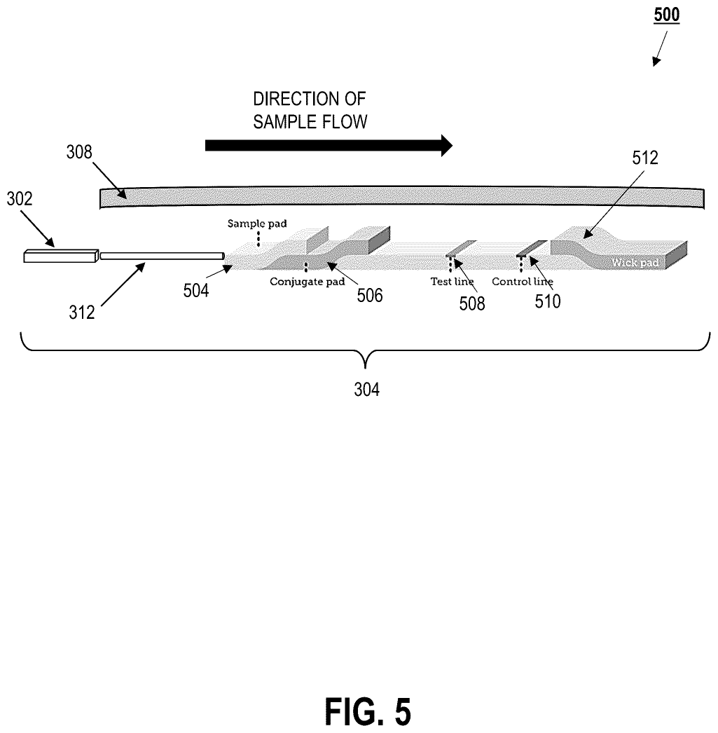

FIG. 5 is a diagram 500 illustrating an exemplary lateral flow (immunoassay) test strip 304 that can be used with the pad shown in FIGS. 3-4, according to some aspects of the disclosed technology. The test strip 304 may include collection pad 302, nanotubing 310, sample pad 504, conjugate pad 506, results rereading area having test line 508 and control line 510, and wick pad 512. Direction of sample flow is illustrated as going from left to right of diagram 500. Transparent polymer layer 308 may be provided over a portion of test strip 304, but leaves at least one or more regions of collection pad 302 exposed or uncovered.

Menses can fall into collection pad 302. Only a small amount of menses is needed to conduct the test.

Nanotubing 310 filled with a buffer can lyse the menses and extract E6 oncoproteins in the user's body from the HPV, and move to the sample pad 504 of test strip 304. In some embodiments, nanotubing 310 may be omitted.

In some cases, nanotubing 310 may include carbon-based nanotubes or nanostructures that may facilitate capillary action of the menses. In some cases, nanotubing 310 may include a buffer. In some cases, nanotubing 310 may be replaced with microfluidic structures. In some cases, nanotubing 310 may be replaced with a fabric or material structure whose fibers and/or filaments are arranged and/or woven to create three-dimensional structures, channels, and/or microgrooves. In some cases, nanotubing 310 may be replaced with materials with hollow cores (e.g., straw-like materials).

The blood sample collects in the test strip 304's sample pad 504, then wicks to the conjugate pad 506, which can include anti-E6 antibodies. If HPV is present, the E6 proteins will bind.

This test strip 304 works similar to the ones found in at-home COVID test and pregnancy tests. The bound pair of E6 antibodies can bind with target antigens on the test line 508, triggering color particles that turn the line blue. When the target antibodies are present, two blue lines (or another color) appear at the end of the test, but only one line appears on the control line 510 when target antibodies are absent. Two blue lines may indicate a positive test result.

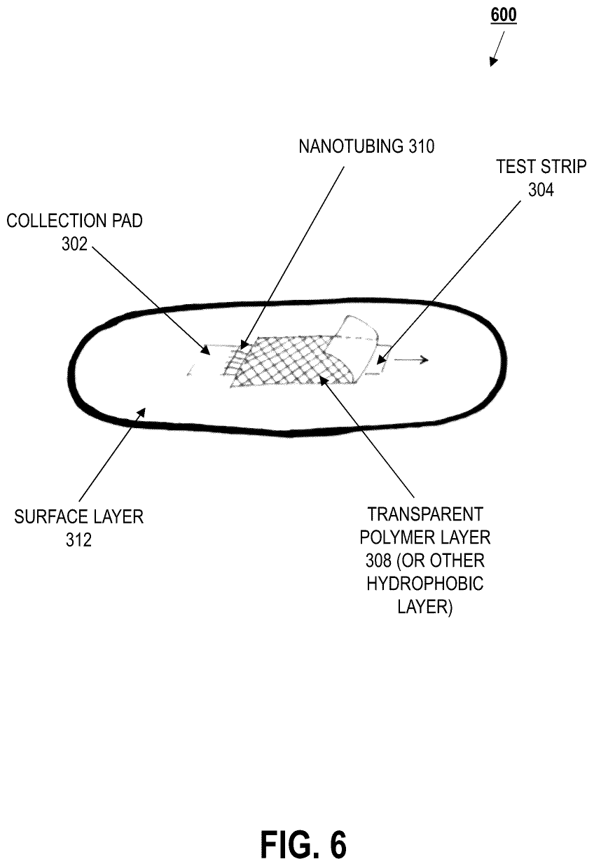

FIG. 6 is a diagram 600 illustrating an exemplary cover (e.g., transparent polymer layer 308) over the lateral flow immunoassay test strip 304, according to some aspects of the disclosed technology. Polyisoprene can be a suitable polymer to cover the testing mechanism for its transparent, hydrophobic, durable, flexible, allergen-free, and biodegradable properties. Polyisoprene may be used as transparent polymer layer 308. There may be alternatives to polyisoprene that enhance hemophobic capacity and transparency. Any other polymer that meets the above criteria and can be engineered to improve surface quality would suffice as a cover in place of transparent polymer layer 308, such as through parchmentization.

In some cases, polymers such as aliphatic polyesters that are not necessarily transparent, but are hydrophobic and biodegradable, can be designed as a covering with a pocket, as seen in FIG. 6. In some cases, the test strip 304 can be removed and read, rather than through a transparent textile.

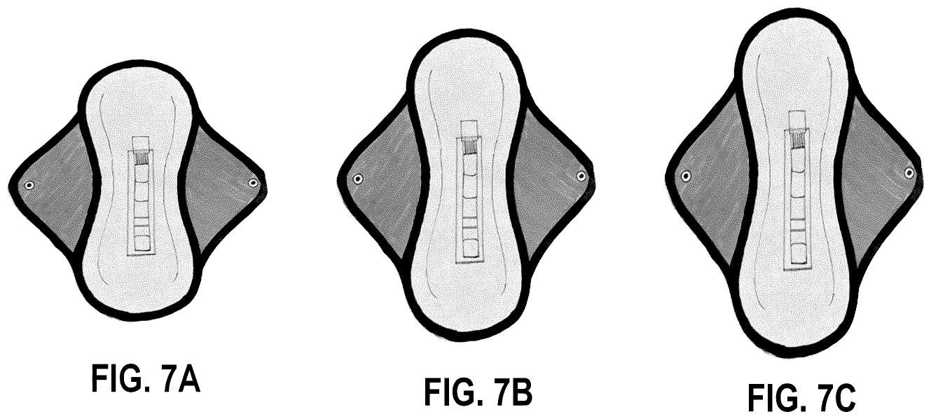

FIGS. 7A-C are top views of exemplary personal care products having different sizing, according to some aspects of the disclosed technology. The length can vary, e.g., between 6-12 inches long. Sizing can also be varied.

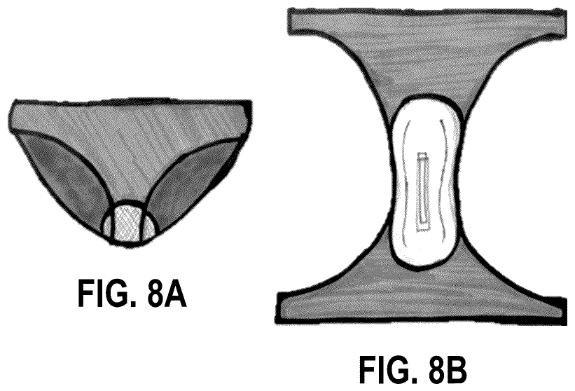

FIGS. 8A-B illustrate integration of diagnostics into a garment, according to some aspects of the disclosed technology. Wing clasps may be replaced with fabric in a panty form. The layered assembly may be integrated into a variety of washable, reusable, wearable garments, such as underwear, shorts, skorts, bodysuit, tights/leggings, etc.

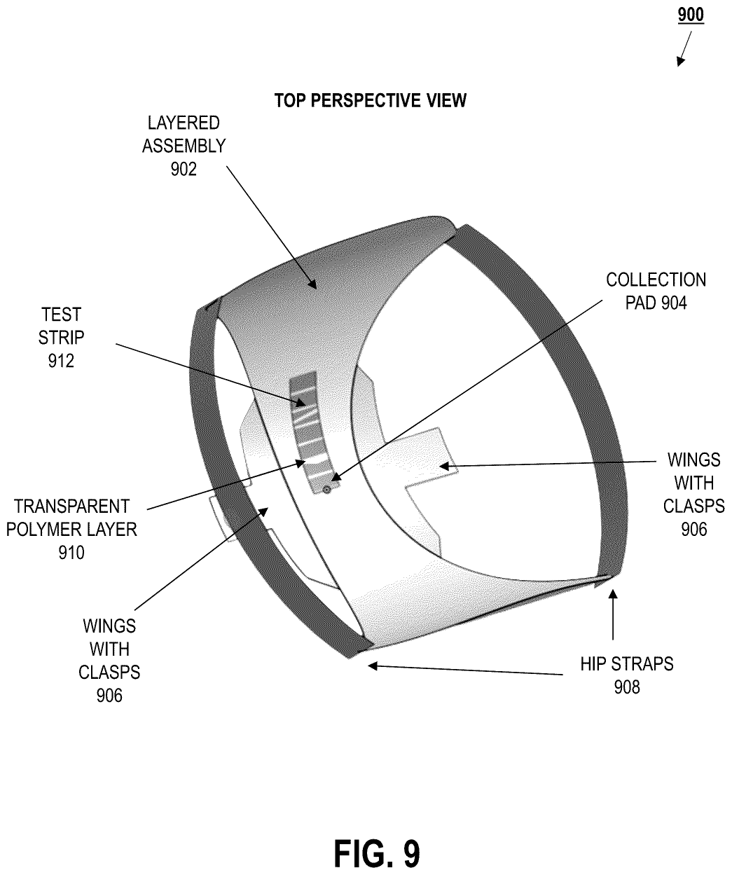

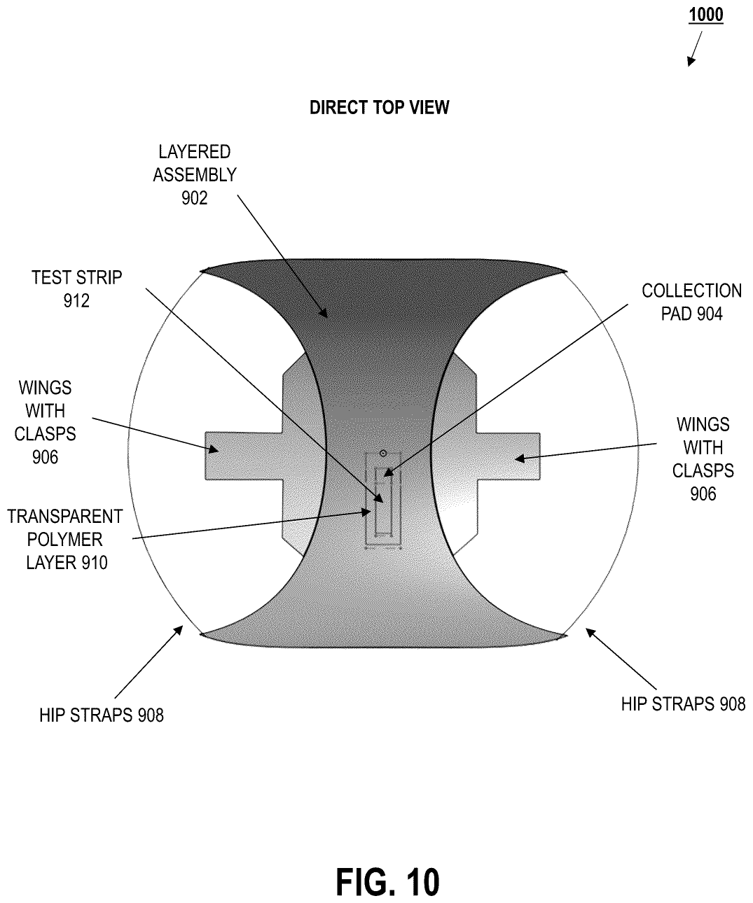

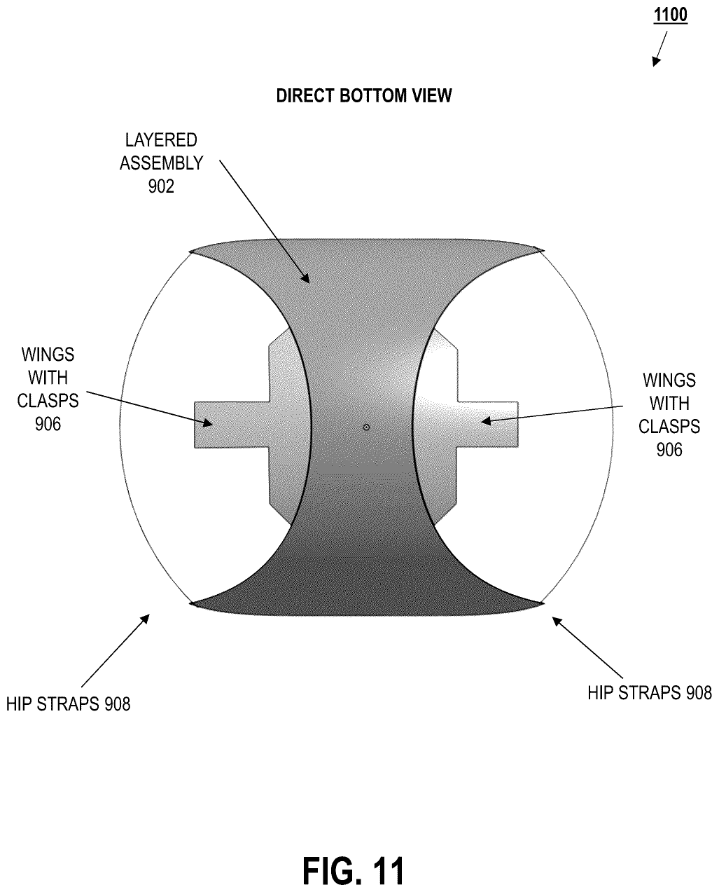

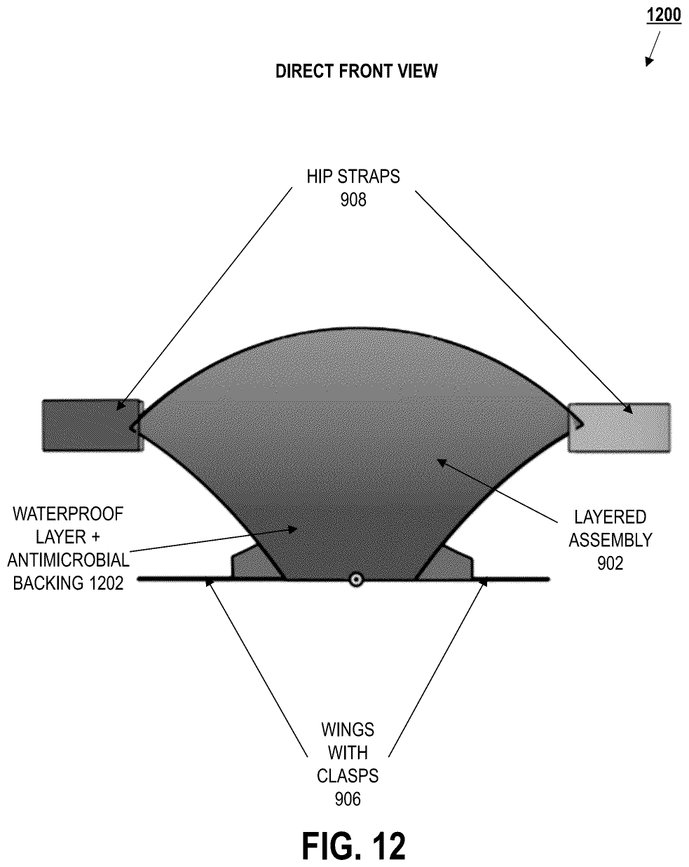

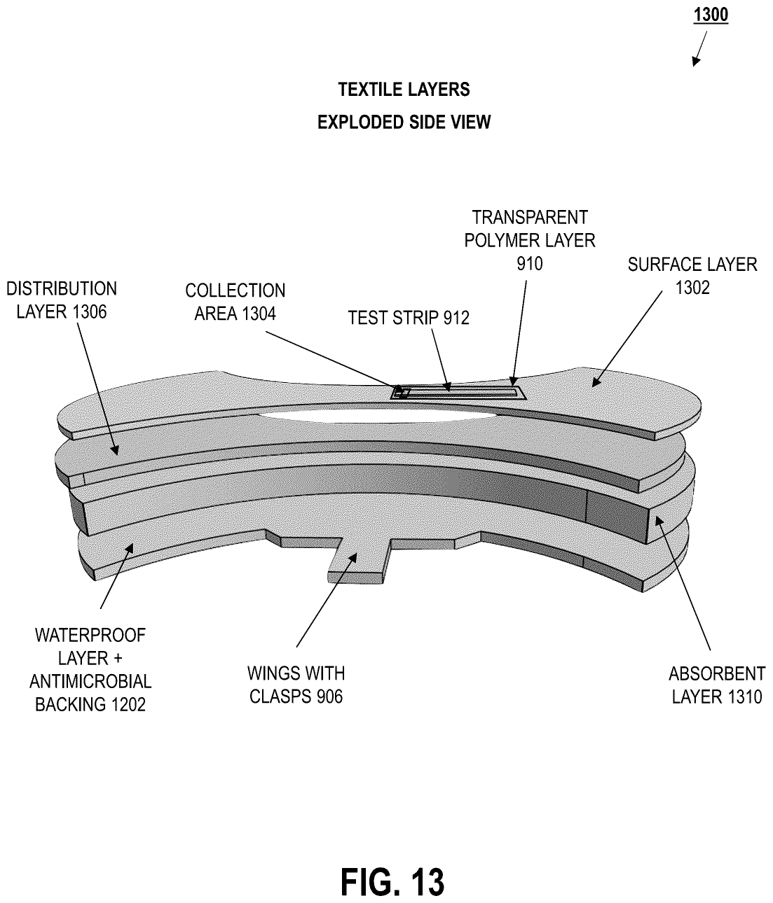

FIGS. 9-12 illustrate different views of a garment having diagnostics embedded therein, according to some aspects of the disclosed technology. FIG. 9 shows a top perspective view 900. FIG. 10 shows a (direct) top view 1000. FIG. 11 shows a (direct) bottom view 1100. FIG. 12 shows a (direct) front view 1200. FIG. 13 illustrates an exploded side view 1300 of layers of layered assembly 902 used in a garment having embedded diagnostics therein, according to some aspects of the disclosed technology. The garment includes hip straps 908 and layered assembly 902. The garment may include wings with clasps 906. The garment includes test strip 912.

The garment may include a constructed (washable and reusable) menstrual pad having a diagnostic mechanism embedded on a surface of the pad. Rather than just being a menses collection device, the garment has a screening mechanism directly attached to its surface. Screening results are provided via the pad within a shorter time frame. The diagnostic testing mechanism is protected by a transparent polymer. Hip straps (e.g., hip straps 908) are attached to layered assembly 902 to ensure precise location of the blood collection while the user partakes in daily activities with maximum comfort. Testing does not interfere with the garment serving its dual purpose as a regular-use menstrual pad. Each textile was intentionally designed.

The garment may include a layered assembly 902. As seen in FIG. 13, layered assembly 902 includes transparent polymer layer 910, surface layer 1302, distribution layer 1306, absorbent layer 1310, and waterproof layer 1202 with antimicrobial backing/coating/layer.

Transparent polymer layer 910 may cover a first portion of test strip 912 to protect the reactive mechanisms that may occur on test strip 912. Transparent polymer layer 910 may expose a second portion of test strip 912 to allow for menses to be collected onto test strip 912. The second portion may include a collection pad 904 of test strip 912.

Transparent polymer layer 910 can have multiple purposes. First, transparent polymer layer 910 prevents the entrance of varying bodily fluids and environmental factors into the test, preserving the integrity of the diagnostic mechanism. Second, the transparent nature allows for visualization of the test results. Lastly, transparent polymer layer 910 secures the test in its intended location. Transparent polymer layer 910 may surround all of the testing mechanism except for where menses is collected for evaluation, or the collection pad 904. Transparent polymer layer 910 may include one or more slits or openings to expose a collection area (e.g., collection area 1304). The slits or openings may be provided to ensure a suitable amount of menses is collected through collection area 1304. The slits or openings may be positioned to direct or channel menses to collection pad 904. Collection area 1304 may include one or more windows in transparent polymer layer 910.

Transparent polymer layer 910 can be made from any material that can provide these functions. Existing materials can also be engineered to meet these purposes. Examples of materials of interest include polyisoprene. Exemplary materials include polyisoprenes, natural rubber, and synthetic rubber. Parchmentization of materials can be used in place of transparent polymer layer 910. Transparent polymer layer 910 may be attached to the pad's surface layer 1302 via any durable method that retains the pad's ability to be washed.

Transparent polymer layer 910 may be bonded to all sides of the test strip 912, or at least a perimeter of test strip 912. Transparent polymer layer 910 may seal edges or perimeter of test strip 912 to surface layer 1302. In some cases, transparent polymer layer 910 may not form a pocket to allow removability. Pocket as used herein describes a partial enclosure (e.g., a sleeve) where the test strip 912 may be removed easily without destroying the pocket. If there is a plastic casing enclosing test strip 912 or test strip 912 is provided as a cassette, transparent polymer layer 910 can be used to hold this cassette down and in place.

Transparent polymer layer 910 may seal a perimeter or an area surrounding test strip 912 to surface layer 1302 to prevent test strip 912 from being removable and keeps test strip 912 in place.

In some cases, test strip 912 may be on top of surface layer 1302 but under transparent polymer layer 910. For a smooth top surface of layered assembly 902 facing a sensitive area of the user, test strip 912 may be provided at least partially embedded in surface layer 1302. In some cases, test strip 912 may be provided at least partially below surface layer 1302. In some cases, test strip 912 may be on top of distribution layer 1306.

Layered assembly 902 may include biodegradable textiles. Textiles may be selected with user comfort in mind. Textiles may be selected for their moisture wicking properties. Textiles may be selected for absorbency. Surface layer 1302 may include soybean-based fabric. Surface layer 1302 may include organic cotton. Distribution layer 1306 may include Lyocell (having wood cellulose and synthetic substances). Distribution layer 1306 may include linen. Distribution layer 1306 may include fibers which create a directional structure to direct fluid towards absorbent layer 1310. Absorbent layer 1310 may include Zorb fabric (having a blend of cellulosic fibers with synthetic fibers). Zorb fabric may be produced by AKAS Tex. Absorbent layer 1310 may include organic cotton. Absorbent layer 1310 may include banana fabric. Absorbent layer 1310 may include bamboo-fiber fabric. Waterproof layer 1202, on one surface, may be treated with a hydrophobic finish. Waterproof layer 1202 on another surface, may be treated with Microban. Layered assembly 902 may use sustainable fabrics, which are beneficial to the environment (as opposed to plastic-based materials). Layered assembly 902 is washable, cost-effective, and functional.

Diagnostics such as test strip 912 (e.g., a lateral flow assay test strip) may be embedded in layered assembly 902. Test strip 912 may be embedded in layered assembly 902 in a manner to ensure a smooth top surface, which may face a user's sensitive skin during use. Test strip 912 may include collection pad 904. Test strip 912 may be embedded in layered assembly 902 by means of transparent polymer layer 910.

Layered assembly 902 may be designed to offer front, bottom, and back coverage of a user's when worn by the user. Layered assembly 902 that covers a user from a front of the user just below the abdomen to the back of the user up to the abdomen. Layered assembly 902 may, when the garment is worn by a user, extend from below the user's abdomen, across the user's groin, and to the user's lower back. Layered assembly 902 may provide coverage from the pelvic region to the upper buttocks region of a user when worn.

The garment includes hip straps 908 that wraps a user on the left side and the right side of the hip. The hip straps are affixed (scaled, stitched, heat bonded, and/or sewn) to the layered assembly 902. Hip straps 908 extends horizontally. Hip straps 908 hang on a user's hip when the garment is worn by the user. Sizing of the garment may be adjusted by adjusting the length of hip straps by moving a slide adjustor. Hip straps 908 attach directly to the layered assembly 902. Hip straps 908 are adjustable to accommodate user body types and are made with environmentally-conscious and/or biodegradable materials. Hip straps 908 may be adjustable in length. Hip straps 908 may include a first hip strap and a second hip strap. The first hip strap can extend from a first corner of the layered assembly 902 to a second corner of the layered assembly 902. The second hip strap can extend from a third corner of the layered assembly 902 to a fourth corner of the layered assembly 902. Hip straps 908 may be adjustable in length using extensions. Hip straps 908 cannot be taken off or are not intended to be detached from layered assembly 902. It is important to ensure that test strip 912 maintains its location and is minimally affected by any friction from the user's daily activities. Hip straps 908 may help to stabilize the garment on the user to ensure test strip 912 maintains its location relative to the user while the user is wearing the garment.

In comparison to a sanitary belt, these hip straps 908 do not extend down. Rather, the layered assembly 902 is simply longer, thus has a different look and functionality all together.

The garment may include wings with clasps 906 (e.g., winged portions with clasp enclosures) to allow the wings to close and wrap around another piece of garment. Clasp enclosures may include snap clasps, buttons, eco-friendly buttons, and other suitable clasps.

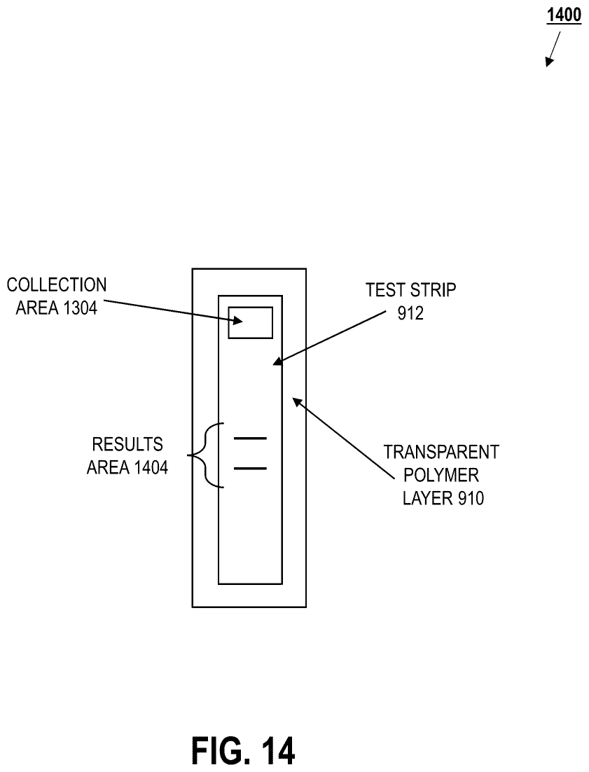

FIG. 14 illustrates a top view 1400 of embedded diagnostics and display of diagnostics results, according to some aspects of the disclosed technology. Test strip 912 is partially covered by transparent polymer layer 910 and partially exposed by transparent polymer layer 910. The area of test strip 912 exposed by transparent polymer layer 910 is shown as collection area 1304. Target analytes may react with certain chemicals in results area 1404, which may result in a visual change in results area 1404 (e.g., show two lines).

Results seen through results area 1404 are not meant to be received through a third party source, such as a lab. Results will be conveyed to the user directly, via reactions on the test triggering colors to appear on the test. This may take different forms. With a sandwich ELISA or LFA, two lines would appear. Color forming on the test line and control line indicates the presence of the analyte. Results are private to the user viewing the results.

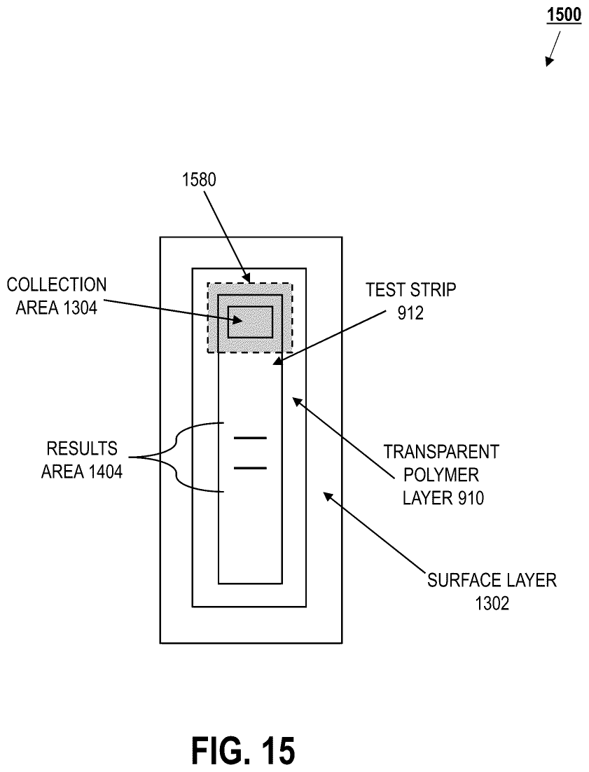

FIG. 15 illustrates a top view 1500 of embedded diagnostics and display of diagnostics results and a part of the layered assembly 902, according to some aspects of the disclosed technology. In some cases, layered assembly 902 may further include a removable layer 1580 that can cover one or more openings of the transparent polymer layer 910 that exposes the second portion of the test strip 912. The removable layer 1580 may be adhered to the transparent polymer layer 910 and/or the surface layer 1302 through static electricity. In some cases, the removable layer 1580 may include parafilm. The removable layer 1580 may be adhered to the transparent polymer layer 910 and/or the surface layer 1302 through a weak adhesive. In some cases, the removable layer 1580 may include a hook and loop mechanism for attaching to the surface layer 1302. In some cases, the removable layer 1580 may be removed to expose the second portion of the test strip 912 by a user. Before removal the removable layer 1580 may protect the second portion of the test strip 912 from contaminants. In some cases, the removable layer 1580 may be reattached to the transparent polymer layer 910 and/or the surface layer 1302 by a user to cover the one or more openings of the transparent polymer layer 910 that exposes the second portion of the test strip 912.

In some cases, a different cover or patch that may be used by a user to cover the one or more openings of the transparent polymer layer 910 that exposes the second portion of the test strip 912. The cover or patch may include the same material as the surface layer 1302 on one side and an adhesion mechanism on another side. The cover or patch may be attached to the pad or garment after the diagnostics have been used (e.g., collection of menses was completed) to seal the used diagnostics mechanism securely in the pad or garment. In this manner, the cover or patch can ensure that the diagnostics do not fall out during washing or rewearing of the pad or garment. The adhesion mechanism of the cover or patch may allow the cover or patch to adhere securely to adhered to the transparent polymer layer 910 and/or the surface layer 1302. The adhesion mechanism may include a strong fabric adhesive. The adhesion mechanism may include a hook and loop mechanism. The cover or patch may include a fabric sticker. The cover or patch may include fabric adhesive tape.

One method of using the pads and/or garments described herein includes:
A user removes removable layer 1580 and wears pad on day 1 of menstrual cycle.
Menses is collected by the diagnostics embedded in the layered assembly.
User reattaches removable layer 1580 and/or cover/patch to cover collection pad of test strip and/or seal the test strip inside the layered assembly.
User may wash or wipe surface of pad when full.
Looking through transparent polymer layer for test results, such as two blue lines,
Thoroughly wash pad,
Rewear pad on a subsequent day of menstrual cycle, and
After washing pad, the test results would not be valid, but pad can be reused, e.g., for additional days of the menstrual cycle and further menstrual cycles not requiring testing.

### Textile Considerations

Most collection devices are meant to be disposed or allow for a single time use only. However, the pads and/or garments described herein are reusable as a regular sanitary pad and washable. Longevity of the product drives down the cost per use. Because the test is covered in the transparent polymer, washing the pad is safe, and risk of harm from the testing mechanism is minimized. Each textile is chosen for its ability to be absorbent, biodegradable, naturally sources, yet cost efficient. Each layer of the pad serves a purpose to ensure effective moisture wicking and durability. Providing antimicrobial backing is novel and nonobvious. This layer's main purpose is to prevent bacterial growth during use, and between uses when washing.

### Diagnostics Test Strip Designs and Considerations

Various examples herein describe using a lateral flow assay test strip. The proposed diagnostic mechanism can vary based on the analyte of interest and whether the result required is qualitative or quantitative. Solutions include sandwich Enzyme-Linked Immunosorbent Assays (ELIZAS) and other lateral flow devices. The testing strip may be embedded in the pad with or without a plastic casing. The cassette (e.g., testing strip with a plastic casing) would allow for more accurate readings by allowing fluid to flow effectively and provide a protective barrier for the strip.

A buffer solution may be provided with the pad to aid stimulating capillary action on the test strip or may be integrated into the collection region via tubing or grooves. A buffer solution may be stored in a blister pack positioned adjacent to the testing strip to release the buffer when a user presses to open the blister pack. Nanotubing, structured fibers, and/or microfluidic structures may be used to carry the buffer from the blister pack and the menses sample towards a reaction area of the test strip. Buffer solution may be particularly helpful for aiding movement of menses in the testing strip.

### Summary of Features, Benefits, and Other Variations

The following list of biodegradable textiles or combinations/blends thereof can be utilized to create a layer of the pad, considering user comfort and absorbency: Organic Cotton, Hemp, Wool, Silk, Bamboo-Fiber Fabrics, Jute, Ramie, Abaca, etc. In some embodiments, the pads (and/or garments) do not utilize plastic-based fabrics that are both unsustainable to manufacture and non-biodegradable, like polyester, rayon, nylon, acrylic, and fleece, which are commonly used in reusable period products currently. In some embodiments, animal-based textiles may also be avoided as chemical treatments make them unable to biodegrade and unsafe to have in-contact with the vulva.

The pads (and/or garments) described herein offer many benefits. The pads do not use biosensors or electrochemical sensors. Instead, the pads include LFA strips, which are more accessible. The pads offer easier data/test result interpretations. Users can understand the results of the LFA strip and record a color change. No smartphones or transceiver/reader electronics, and reliable Internet/wireless connections are required. The tests offered by the pads are lab-free. The pad is constructed with washable and reusable textiles. Users can repeatedly wash and rewear the pad per the users' convenience, without risk of damaging sensitive diagnostic technologies in the pad.

The pad can be adjusted to test for a variety of analytes, utilizing other polymer coverings, being constructed by other textiles, or expanding its sizes.

Examples of biomarkers are shown in the table below:

| Biomarkers | Examples |
|---|---|
| Vitamins & | D, B12, iron, potassium, |
| Minerals | magnesium, sodium |
| Infectious | chlamydia, HIV, syphilis, |
| Disease | herpes, T-cells |
| Cardiovascular | cholesterol, triglycerides |
| Cancer | 15-3 (breast), 125 (ovarian or fallopian) |
| Fertility | estrogen, progesterone, FSH |
| Blood | hemoglobin, HbA1c |
| Organs | creatinine, ALAT, ASAT |

The LFA strip in the pads can be designed to screen for a variety of biomarkers in menses by altering the antibodies in the conjugate pad that bind to the desired antigens/proteins in the sample. The LFA's color particles can be triggered either by presence of the protein (for infectious diseases or cancer biomarkers) or by an amount of protein that is less than or greater than what is healthy (for vitamins, cardiovascular biomarkers, or for the health of other organ systems). The quantity of buffer can change based on concentrations of biomarkers. Amount of the buffer can be chosen to not overwhelm the test with extraneous material, or to cause a false positive or false negative.

The pads with an integrated LFA strip may have one or more of the following advantages:
Low-Cost: Easier and more inexpensive to produce than electrochemical sensors
User Comfort: Easy to make compact and flexible
Rapid: Requiring only between 5-15 minutes for the analyte to be lysed in the buffer, and another 5-15 for results to be processed
Manufacturing: Already commonly used for variety of viral/infectious diagnostics, so easier to manufacture for other concerns
Safe: Pads can be washed and reused without interfering with diagnostics or risking user safety
Accessible: Doesn't require a lab or external facility to analyze results, everything can be conducted by the user at home
User-Friendly: No need for an internet/Bluetooth connection or a smartphone/personal device to give results to the users

The pad structure may have one or more of the following advantages:
Flexible: Can be constructed to fit a specific user or region's needs
Eco-Friendly: Each textile, including the polymer covering, is environmentally conscious; quick to biodegrade and easy to manufacture or access
Reusable: With increasing period poverty rates in developing regions, users receive both diagnostics and a hygiene product, that they can wash and reuse
Pathogen-Resistant: Materials are quick to dry and use natural anti-microbial methods that protect users from infections
Non-Invasive: The entirety of the pad serves as a sample collection method for diagnostics, rather than relying on invasive drawings of blood
User Comfort: Clasps allow easy, leak-free wear of the hygiene product. Variations in size or structure accommodate for a variety of menstruating people's needs

In some cases, the pad structure may have one or more of the following advantages:
Low-Cost: Easier and more inexpensive to produce than electrochemical sensors, and uses textiles that are easy to source and allow reusability
Accessible: Doesn't require a lab or external facility to analyze results, everything can be conducted by the user at home. No need for an internet/Bluetooth connection or a smartphone/personal device to give results to the users. Results provided quicker.
User Comfort: Hip straps allow easy, leak-free wear of the hygiene product while maintaining test integrity. Variations in size or structure accommodate for a variety of menstruating people's needs. Garment may include coatings/backings/layers/fabrics with antimicrobial properties.
Manufacturing: Already commonly used for variety of viral/infectious diagnostics, so easier to manufacture for other concerns. Test can be easily altered to seek or test for other analytes.

Several solutions described herein alleviates many concerns, and offers many other benefits and takes into other considerations, including, but not limited to, reusability, washability, cost, lab-free, accessibility to materials, easy to manufacture or make, safe against human skin, comfortable to wear, sustainability, low environmental impact, case of use, etc.

Some aspects described herein apply to a feminine product, e.g., a menstrual pad. However, it is envisioned by the disclosure that the aspects can be applied to and/or integrated into other feminine/menstrual hygiene products such as period panties, etc. It is envisioned by the disclosure that the aspects can be applied to and/or integrated into other personal care products such as diapers or under garments for babies, toddlers, adults, pets, etc. It is envisioned by the disclosure that the aspects can be applied to and/or integrated into under garments for babies, toddlers, adults, pets, etc. In addition, it is envisioned by the disclosure that the aspects can be applied to and/or integrated into clothing or garments such as jumpsuits, onesies, overalls, pantyhose, shorts, or pants for babies, toddlers, adults, pets, etc.

The LFA strip can be used to test for presence of one or more markers and is not limited to testing for HPV. The LFA strip can be constructed to test for the presence of different markers (e.g., a panel of markers).

### SELECT EXAMPLES

Example 1 provides a garment, including a layered assembly having a translucent polymer layer, a surface layer, distribution layer, an absorbent layer, and a waterproof layer with an antimicrobial backing; a lateral flow assay test strip embedded in the layered assembly, where the translucent polymer layer covers a first portion of the lateral flow assay test strip, and the translucent polymer layer exposes a second portion of the lateral flow assay test strip; a first hip strap extending from a first corner of the layered assembly to a second corner of the layered assembly; and a second hip strap extending from a third corner of the layered assembly to a fourth corner of the layered assembly.

Example 2 provides the garment of example 1, further including wings; and clasps for closing wings.

Example 3 provides the garment of example 1 or 2, where the layered assembly, when the garment is worn by a user, extends from below the user's abdomen, across the user's groin, and to the user's lower back.

Example 4 provides the garment of any one of examples 1-3, where the first hip strap and the second hip strap, when the garment is worn by a user, hang on the user's hips.

Example 5 provides the garment of any one of examples 1-4, where the translucent polymer layer is bonded to the surface layer and the lateral flow assay test strip to seal the lateral flow assay test strip in the layered assembly.

Example 6 provides the garment of any one of examples 1-5, where the lateral flow assay test strip is enclosed in a plastic casing.

Example 7 provides the garment of any one of examples 1-6, where the first hip strap and the second hip strap are adjustable in length.

Example 8 provides the garment of any one of examples 1-7, where the translucent polymer layer is made from a hydrophobic polymer.

Example 9 provides the garment of any one of examples 1-8, where the translucent polymer layer is made from polyisoprene.

Example 10 provides the garment of any one of examples 1-9, where the second portion of the lateral flow assay test strip includes a collection pad at a first end of the lateral flow assay test strip.

Example 11 provides the garment of any one of examples 1-10, where the lateral flow assay test strip includes a collection pad; a sample pad; and nanotubing fluidically connecting the collection pad to the sample pad.

Example 12 provides the garment of example 11, where the lateral flow assay test strip further includes conjugate pad; results reading area having a test line and a control line; and wick pad.

Example 13 provides the garment of example 11 or 12, where the nanotubing is filled with a buffer.

Example 14 provides a menstrual pad, including a layered assembly having a translucent polymer layer, a surface layer, an absorbent layer, a waterproof layer, and anti-microbial backing layer; a lateral flow assay test strip embedded in the layered assembly, where the translucent polymer layer covers a first portion of the lateral flow assay test strip, and the translucent polymer layer exposes a second portion of the lateral flow assay test strip; wings; and clasps for closing wings.

Example 15 provides the menstrual pad of example 14, where the translucent polymer layer is made from a hydrophobic polymer.

Example 16 provides the menstrual pad of example 14 or 15, where the translucent polymer layer is made from polyisoprene.

Example 17 provides the menstrual pad of any one of examples 14-16, where the second portion of the lateral flow assay test strip includes a collection pad at a first end of the lateral flow assay test strip.

Example 18 provides the menstrual pad of any one of examples 14-17, where the lateral flow assay test strip includes a collection pad; a sample pad; and nanotubing fluidically connecting the collection pad to the sample pad.

Example 19 provides the menstrual pad of example 18, where the lateral flow assay test strip further includes conjugate pad; results reading area having a test line and a control line; and wick pad.

Example 20 provides the menstrual pad of example 18 or 19, where the nanotubing is filled with a buffer.

Example 21 provides a menstrual pad, including a layered assembly having a translucent organdy fabric layer, an absorbent fabric layer, a waterproof layer, and an antimicrobial fabric layer; a lateral flow assay test strip embedded in the layered assembly, where the translucent organdy fabric layer covers a first portion of the lateral flow assay test strip, and the translucent organdy fabric layer exposes a second portion of the lateral flow assay test strip; wings; and clasps for closing the wings.

Example 22 provides the menstrual pad of example 21, where the translucent organdy fabric layer is hydrophobic.

Example 23 provides the menstrual pad of example 21 or 22, where the absorbent fabric layer includes a microfiber fabric material.

Example 24 provides the menstrual pad of any one of examples 21-23, where the waterproof layer includes a polyurethane laminate layer.

Example 25 provides the menstrual pad of any one of examples 21-24, where the antimicrobial fabric layer includes a charcoal bamboo fiber fabric layer.

Example 26 provides the menstrual pad of any one of examples 21-25, where the antimicrobial fabric layer covers the layered assembly and the wings.

### Variations and Other Notes

Although the operations of the example methods shown in and described with reference to the FIGS. are illustrated as occurring once each and in a particular order, it will be recognized that the operations may be performed in any suitable order and repeated as desired. Additionally, one or more operations may be performed in parallel. Furthermore, the operations illustrated in the FIGS. may be combined or may include more or fewer details than described.

The above description of illustrated implementations of the disclosure, including what is described in the Abstract, is not intended to be exhaustive or to limit the disclosure to the precise forms disclosed. While specific implementations of, and examples for, the disclosure are described herein for illustrative purposes, various equivalent modifications are possible within the scope of the disclosure, as those skilled in the relevant art will recognize. These modifications may be made to the disclosure in light of the above detailed description.

For purposes of explanation, specific numbers, materials and configurations are set forth in order to provide a thorough understanding of the illustrative implementations. However, it will be apparent to one skilled in the art that the present disclosure may be practiced without the specific details and/or that the present disclosure may be practiced with only some of the described aspects. In other instances, well known features are omitted or simplified in order not to obscure the illustrative implementations.

Further, references are made to the accompanying drawings that form a part hereof, and in which are shown, by way of illustration, embodiments that may be practiced. It is to be understood that other embodiments may be utilized, and structural or logical changes may be made without departing from the scope of the present disclosure. Therefore, the following detailed description is not to be taken in a limiting sense.

Various operations may be described as multiple discrete actions or operations in turn, in a manner that is most helpful in understanding the disclosed subject matter. However, the order of description should not be construed as to imply that these operations are necessarily order dependent. In particular, these operations may not be performed in the order of presentation. Operations described may be performed in a different order from the described embodiment. Various additional operations may be performed or described operations may be omitted in additional embodiments.

For the purposes of the present disclosure, the phrase “A or B” or the phrase “A and/or B” means (A), (B), or (A and B). For the purposes of the present disclosure, the phrase “A, B, or C” or the phrase “A, B, and/or C” means (A), (B), (C), (A and B), (A and C), (B and C), or (A, B, and C). The term “between,” when used with reference to measurement ranges, is inclusive of the ends of the measurement ranges.

The description uses the phrases “in an embodiment” or “in embodiments,” which may each refer to one or more of the same or different embodiments. The terms “comprising,” “including,” “having,” and the like, as used with respect to embodiments of the present disclosure, are synonymous. The disclosure may use perspective-based descriptions such as “above,” “below,” “top,” “bottom,” and “side” to explain various features of the drawings, but these terms are simply for ease of discussion, and do not imply a desired or required orientation. The accompanying drawings are not necessarily drawn to scale. Unless otherwise specified, the use of the ordinal adjectives “first,” “second,” and “third,” etc., to describe a common object, merely indicates that different instances of like objects are being referred to and are not intended to imply that the objects so described must be in a given sequence, either temporally, spatially, in ranking or in any other manner.

In the following detailed description, various aspects of the illustrative implementations will be described using terms commonly employed by those skilled in the art to convey the substance of their work to others skilled in the art.

The terms “substantially,” “close,” “approximately,” “near,” and “about,” generally refer to being within +/−20% of a target value as described herein or as known in the art. Similarly, terms indicating orientation of various elements, e.g., “coplanar,” “perpendicular,” “orthogonal,” “parallel,” or any other angle between the elements, generally refer to being within +/−5-20% of a target value as described herein or as known in the art.

In addition, the terms “comprise,” “comprising,” “include,” “including,” “have,” “having” or any other variation thereof, are intended to cover a non-exclusive inclusion. For example, a method, process, or device, that comprises a list of elements is not necessarily limited to only those elements but may include other elements not expressly listed or inherent to such method, process, or device. Also, the term “or” refers to an inclusive “or” and not to an exclusive “or.”

The systems, methods and devices of this disclosure each have several innovative aspects, no single one of which is solely responsible for all desirable attributes disclosed herein. Details of one or more implementations of the subject matter described in this specification are set forth in the description and the accompanying drawings.

1. A garment, comprising:
a layered assembly having a translucent polymer layer, a surface layer, distribution layer, an absorbent layer, and a waterproof layer with an antimicrobial backing;
a lateral flow assay test strip embedded in the layered assembly, wherein the translucent polymer layer covers a first portion of the lateral flow assay test strip, and the translucent polymer layer exposes a second portion of the lateral flow assay test strip;
a first hip strap extending from a first corner of the layered assembly to a second corner of the layered assembly; and
a second hip strap extending from a third corner of the layered assembly to a fourth corner of the layered assembly.

2. The garment of claim 1, further comprising:
wings; and
clasps for closing wings.

3. The garment of claim 1, wherein the layered assembly, when the garment is worn by a user, extends from below the user's abdomen, across the user's groin, and to the user's lower back.

4. The garment of claim 1, wherein the first hip strap and the second hip strap, when the garment is worn by a user, hang on the user's hips.

5. The garment of claim 1, wherein the translucent polymer layer is bonded to the surface layer and the lateral flow assay test strip to seal the lateral flow assay test strip in the layered assembly.

6. The garment of claim 1, wherein the lateral flow assay test strip is enclosed in a plastic casing.

7. The garment of claim 1, wherein the first hip strap and the second hip strap are adjustable in length.

8. The garment of claim 1, wherein the translucent polymer layer is made from a hydrophobic polymer.

9. The garment of claim 1, wherein the translucent polymer layer is made from polyisoprene.

10. The garment of claim 1, wherein the second portion of the lateral flow assay test strip includes a collection pad at a first end of the lateral flow assay test strip.

11. The garment of claim 1, wherein the lateral flow assay test strip includes:
a collection pad;
a sample pad; and
nanotubing fluidically connecting the collection pad to the sample pad.

12. The garment of claim 11, wherein the lateral flow assay test strip further includes:
conjugate pad;
results reading area having a test line and a control line; and
wick pad.

13. The garment of claim 11, wherein the nanotubing is filled with a buffer.

14. A menstrual pad, comprising:
a layered assembly having a translucent polymer layer, a surface layer, an absorbent layer, a waterproof layer, and anti-microbial backing layer;
a lateral flow assay test strip embedded in the layered assembly, wherein the translucent polymer layer covers a first portion of the lateral flow assay test strip, and the translucent polymer layer exposes a second portion of the lateral flow assay test strip;
wings; and
clasps for closing wings.

15. The menstrual pad of claim 14, wherein the translucent polymer layer is made from a hydrophobic polymer.

16. The menstrual pad of claim 14, wherein the translucent polymer layer is made from polyisoprene.

17. The menstrual pad of claim 14, wherein the second portion of the lateral flow assay test strip includes a collection pad at a first end of the lateral flow assay test strip.

18. A menstrual pad, comprising:
a layered assembly having a translucent organdy fabric layer, an absorbent fabric layer, a waterproof layer, and an antimicrobial fabric layer;
a lateral flow assay test strip embedded in the layered assembly, wherein the translucent organdy fabric layer covers a first portion of the lateral flow assay test strip, and the translucent organdy fabric layer exposes a second portion of the lateral flow assay test strip;
wings; and
clasps for closing the wings.

19. The menstrual pad of claim 18, wherein the antimicrobial fabric layer comprises a charcoal bamboo fiber fabric layer.

20. The menstrual pad of claim 18, wherein the antimicrobial fabric layer covers the layered assembly and the wings.
